(19) United States
(12) Patent Application Publication
(10) Pub. No.: US 2020/0353174 A1
(43) Pub. Date: Nov. 12, 2020

(54) PEN INJECTOR WITH DRIVE MEMBER AND REDUCER ARM SET

(71) Applicants: Alwin Jogin, Bengaluru (IN); Alok Das, Bengaluru (IN); Jasraj Singh, Bengaluru (IN); Prasad Ghansham Satam, Hyderabad (IN); Nagaprasad Vishnubhotla, Hyderabad (IN); Sivakumaran Meenakshisunderam, Hyderabad (IN)

(72) Inventors: Alwin Jogin, Bengaluru (IN); Alok Das, Bengaluru (IN); Jasraj Singh, Bengaluru (IN); Prasad Ghansham Satam, Hyderabad (IN); Nagaprasad Vishnubhotla, Hyderabad (IN); Sivakumaran Meenakshisunderam, Hyderabad (IN)

(73) Assignee: Aurobindo Pharma Ltd, Hyderabad (IN)

(21) Appl. No.: 16/870,840

(22) Filed: May 8, 2020

(30) Foreign Application Priority Data
May 9, 2019 (IN) 201941018578

(51) Int. Cl. A61M 5/315

(52) U.S. Cl. CPC A61M 5/31526; A61M 5/31585; A61M 2005/3125; A61M 5/31593; A61M 5/31535

(57) ABSTRACT

A portable self-administrable medication delivery device with “End of Life” feature includes a housing, a drive member within said housing, a locking element and a medicine filled container. The device further provides a “Partial Dose Prevention” feature to deliver an accurate amount of dose with precision. The device is capable of delivering multiple doses of a liquid medicament contained therein without the need of priming the injector prior to administration and allows for repeated administration of a dose of medicament in a simple, easy, safe and accurate manner.

### CROSS REFERENCE TO RELATED APPLICATIONS

This application claims priority from an Indian Patent Application IN 201 941 01 8578 filed on May 9, 2019.

### FIELD OF THE INVENTION

The present invention pertains to a medication delivery device, and, in particular, to a portable medication delivery device such as pen injectors. The present invention relates to a pen injector capable of delivering multiple doses of a liquid medicament contained therein without the need of priming the injector prior to administration and allows for repeated administration of a dose of medicament in a simple, easy, safe and accurate dosing to patients by self-administration.

### BACKGROUND OF THE INVENTION

Usually pen injectors must provide an effective delivery of drugs or biological products, and they should enhance safety, improve dosing accuracy, and increase patient compliance, particularly in self-administration settings. For example, these injectors are required to be designed to provide an accurate method of injecting a dose of drug/biological product contained in a cartridge through an automatically or manually inserted hypodermic needle(s). They are intended for use by a healthcare provider or for self-administration by a patient. Injectors may be designed for single use or multiple uses, and may be disposable or reusable. For example, a single use injector may be used in acute intervention for treatment or prevention while a multi-dose injector may be used as part of a single patient's long term treatment regimen. Normally the multi-dose pen injectors are meant for different and varied types of drug treatments, including but not limited to antidiabetic drug therapy, hormonal therapy and the like, require administration of the drug-contained as liquid medicament at regular intervals over a prolonged period of time. Pen injectors are essentially sophisticated “cartridge-based” syringes. The first pens were introduced for the reliable and accurate self-administration of first-wave of biotech molecules, mainly insulin and human growth hormone (hGH). These therapies require frequent, often daily, manual injection with weight-based or fixed dosing and injections are repeated until the prescribed period or duration—usually 1-2 weeks or up to 1 month.

Pen injectors today must have a high level of convenience and provide the consistent and accurate drug dose delivery for patients.

1. Accommodate readily available pre-filled cartridges for reusable devices

2. Light weight and portable for hand carry on self, during the prescription period

Current Inventions
I. For example geared-driven injection mechanics is taught by U.S. Pat. No. 7,857,791 of Eli Lilly and Company covering a medication dispensing apparatus comprising: a housing; a drive member comprising a plunger movable relative to said housing from a distal position to a proximal position; a fluid container with a rubber pad that is advanceable by the drive member when such drive member is moved distally by a driving means for interconnecting said drive member and said plunger includes a gear set comprising a first pinion in meshed engagement with a rack of said plunger and a second pinion in meshed engagement with a rack of said drive member; and characterized in that at least a portion of said drive member extends through an opening through at least one of said first and second pinions, said opening extending completely through a diameter of said at least one of said first and second pinions.
II. Another example of a geared-driven injection mechanics associated with a locking feature enabled by administration of final dose is taught by U.S. Pat. No. 7,517,334 of Eli Lilly and Company covering a medication dispensing apparatus with a spring-driven locking feature including a latching element having a skid that is slidable along a surface of the drive member as the drive member passes distally during advancement. The drive member is arranged with the skid so as to maintain a latching lip of the latching element against a spring force in a first position free of the driving means during dose preparing and injecting prior to a final dose administration. The skid-engaging surface shifts distally of the skid such that the skid passes beyond a proximal end of that surface upon administration of a final dose, whereby the latching lip is urged by the spring force from the first position to a second position to physically lock the driving means to prevent further dose preparing and injecting.
III. Another geared-driven injection mechanics was taught by U.S. Pat. No. 9,707,354 of Antares Pharma, INC covering a dispensing mechanism, comprising: a housing having a proximal-distal axis; a ram within the housing, movable in a distal direction; a user-operable push button moveable along the proximal-distal axis relative to the housing, the push button including a push button slot at a distal portion of the push button; a crank arm having a pawl tooth, a pivot point, and a crank arm engagement member slidably engageable with the push button slot such that movement of the push button causes the crank arm engagement member to move along the push button slot, causing rotation of the crank arm about the pivot point; a ratchet gear having a first set of teeth releasably engageable with the pawl tooth and a second set of teeth releasably engageable with the ram, wherein engagement of the pawl tooth with the first set of teeth of the ratchet gear causes the ratchet gear to rotate, causing the ram to distally advance relative to the housing; and an anti-reverse mechanism including: at least one housing ratchet integrally formed on an internal surface of the housing; and a flexible column integrally formed on and extending from a distal portion of the push button, the flexible column having a flexible column protrusion at a proximal end thereof, wherein as the push button moves along the proximal-distal axis, the flexible column protrusion engages the housing ratchet and restricts movement of the push button to one direction during a resetting motion.

The aforementioned patents teach complex gear-driven injection technology which have certain limitations and drawbacks like being non-user friendly and prone to having greater chances of mechanical damage during use. Since, gear-driven injection technology were proven to be not safe & effective pen injectors for use by patients throughout the drug treatment period, thus there is a long felt need for the use of a simple, easy, ideal and user-friendly injection technology, other than Gear-driven injection technology, which has no step of Priming the injector prior to administration and can be effectively and safely used for several diseases or disorders with any type of drug dosage regimen and/or treatment periods as prescribed by the prescribers. For e.g. the use of a pen injector for a fixed daily dose injections for the specified time period for the treatment of osteoporosis.

### SUMMARY OF INVENTION

The present invention provides a medication delivery device, like a pen injector for a simple, easy, safe and accurate dosing of drugs or biological products.

The present invention provides a medication delivery device, which comprises a housing, a drive member within said housing, a locking element and a medicine filled container.

The present invention provides a pen injector device, which comprises a housing, a drive member within said housing, a locking element and a medicine filled container.

The present medication delivery device comprising a drive member within the said housing which comprises plunger, holding ratchet, drive ratchet and reducer arm set.

The present medication delivery device comprising a locking element comprising an End of Life (EOL) member and a partial dose prevention (PDP) plate.

The present medication delivery device provides a Partial dose prevention (PDP) comprising a partial dose prevention plate, which moves in a one way track.

The present invention provides a medication delivery device comprising: a housing; a drive member within said housing movable in distal direction and proximal direction; a medicine-filled fluid container with a movable rubber pad at one end and an outlet at the other end, said rubber pad engageable by plunger to be advanced toward said outlet a distance equal to a distal movement of said plunger; wherein the drive member comprising of plunger, holding ratchet, drive ratchet and reducer arm set; a locking element comprising of an End of Life (EOL) member and a partial dose prevention (PDP) plate, wherein the said EOL engaged within the upper housing or within the lower housing, in one embodiment within the upper housing and a part of EOL member sliding over the plunger member, and the said sliding EOL member losses the contact with the sliding surface of the plunger member after final dosing and a component of EOL member snaps downward and latches with the PDP plate and thereby blocks further dosing.

The present medication delivery device provides dose setting steps comprising of pulling of Dose knob thumb-pad which in turn pulls the carrier, the carrier which is linked to the fork link further pulls the reducer arm. The reducer arm slides in the drive ratchet, further drive ratchet moves in the proximal direction sliding on the plunger, where the plunger will be held in a position by holding ratchet positioned in the bottom housing to deliver the medication.

The present medication delivery device provides dose setting steps comprising of pulling of Dose knob thumb-pad which in turn pulls the carrier, the carrier which is linked to the fork link further pulls the reducer arm. The reducer arm slides in the drive ratchet, further drive ratchet moves in the proximal direction sliding on the plunger, where the plunger will be held in a position by holding ratchet positioned in the bottom housing, Wherein PDP held in the dose knob slides on the one way track of the upper housing, further the plunger is still held by the holding ratchet and ready for the delivery of the medication.

The present medication delivery device provides Dose delivery steps comprising of pushing the Dose knob Thumb-pad which further pushes carrier linked to the fork link which in turn pushes reducer arm which slides over the drive ratchet. Thereby the drive ratchet pushes the plunger in distal direction to slide over the holding ratchet to push the rubber pad forward which is attached to plunger to deliver predetermined dose.

The present medication delivery device is a disposable injection pen, in that after the quantity of medicament contained therein is exhausted by multiple operations of the medication device, the medication device is discarded rather than being reset and re-used with a replacement container of medicament.

The present medication delivery device can be reset after completion of preset doses and can be further reusable.

The present medication delivery device is a reusable pen, in which the injection device can be re-set and a new medicament cartridge installed after the quantity of medicament contained therein is exhausted.

One advantage of the present invention provides a medication delivery device comprising of a simple locking element which provides a foolproof mechanism for automatically locking the device to prevent further use after a final dose of the device has been administered.

Another advantage of the present invention provides a medication delivery device comprising of a partial dose prevention plate which provides a standard, reliable, and accurate dosing and automatically prevents the partial dosing of drugs or biological products in a simple, easy and safe way according to the recommended dosage regimen and treatment schedule of drug products.

Another advantage of the present invention provides a medication delivery device comprising of a reducer arm set, which provides an accuracy to dose to be administered in a simple, easy and safe way according to the recommended dosage regimen and treatment schedule of the products being administered.

### BRIEF DESCRIPTION OF THE DRAWINGS

FIG. 1 represents a top perspective view of bottom housing assembly of the medication dispensing device;

FIG. 2 represents a top perspective view of the reducer arm set assembly of medication dispensing apparatus;

FIG. 3 is a top perspective view of the partial dose prevention plate with carrier assembly;

FIG. 4 is a top perspective view of a housing half showing End of Life assembly and one way track of partial dose preventing mechanism;

FIG. 5 is a cross section side view of the injection device with all components.

### DETAILED DESCRIPTION OF THE INVENTION

The present invention provides a medication delivery device for simple, easy, safe and accurate dosing of drugs or biological products. In certain embodiments, the medication delivery device is a pen injector.

In certain embodiments, the present invention provides a medication delivery device for a simple, easy, safe and accurate administration of drugs or biological products for different and varied types of drug treatments, including but not limited to antidiabetic drug therapy, hormonal therapy and the like.

In certain embodiments, the present invention provides pen injectors for a simple, easy, safe and accurate administration of a fixed-dose drug administration of drugs or biological products according to the recommended dosage regimen and treatment schedule for different and varied types of drug treatments, including but not limited to antidiabetic drug therapy, hormonal therapy and the like.

Usually various types of drug treatments, require administration of the drug-containing liquid medicament at regular intervals over an extended period of time. For example, a specific hormone treatment require a daily administration of the drug for a certain period of time. In such a situation, it is advantageous to provide a device that allows the patient to self-administer the medicated injection.

In one embodiment, the present invention provides a medication delivery device, which comprises a housing, a drive member within said housing, a locking element and a medicine filled container.

In one embodiment, the present invention provides a medication delivery device such as a pen injector device.

In one embodiment, the present invention provides a pen injector device, which comprises a housing, a drive member within said housing, a locking element and a medicine filled container.

In one embodiment, the medication delivery device of the present invention provides a pen injector device comprising a drive member within the housing which comprises plunger, holding ratchet, drive ratchet and reducer arm set.

In one embodiment, the medication delivery device of the present invention provides a pen injector device comprising a locking element comprising an End of Life (EOL) member and a partial dose prevention (PDP) plate.

In one embodiment, the present medication delivery device is a disposable injection pen, in that after the quantity of medicament contained therein is exhausted by multiple operations of the medication device, the medication device is discarded rather than being reset and re-used with a replacement container of medicament.

In one embodiments, the present medication delivery device can be reset after completion of preset doses and can be further reusable.

In one embodiment, the present medication delivery device is a reusable pen, in which the injection device can be re-set and a new medicament cartridge installed after the quantity of medicament contained therein is exhausted.

In one embodiment, the present invention provides a medication delivery device comprising: a housing; a drive member within said housing movable in a distal or proximal direction or remains stationary; a medicine-filled fluid container with a movable rubber pad at one end and an outlet at the other end, said rubber pad engageable by plunger to be advanced toward said outlet a distance equal to a distal movement of said plunger; wherein the drive member comprising of plunger, holding ratchet, drive ratchet and reducer arm set; a locking element comprising of a EOL member and a PDP plate, wherein the said EOL member engaged within the upper housing and a part of EOL member sliding over the plunger member, and the said sliding EOL member losses the contact with the sliding surface of the plunger member after final dosing and a component of EOL member snaps downward and latches with the PDP plate member and thereby blocks further dosing.

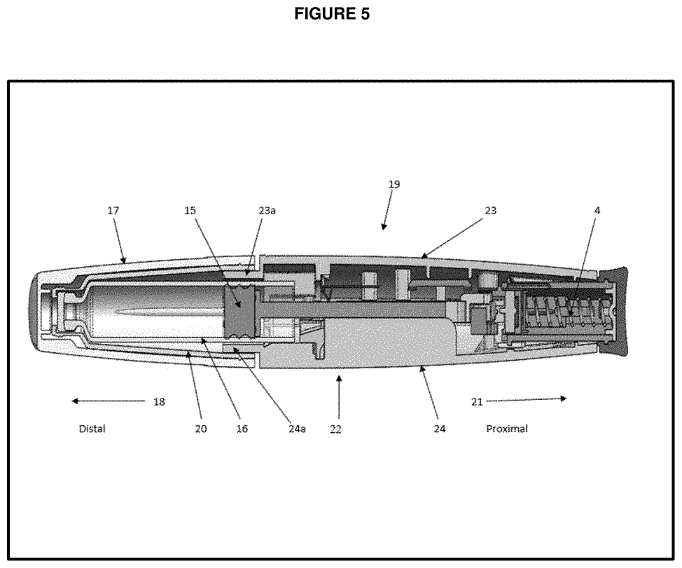

In one embodiment, the medication delivery device is a pen injector comprising a distal and a proximal portion as shown under FIG. 5, wherein the distal portion of the pen injector includes a plastic tubular retainer 20 that holds a cartridge 16 therein. Cartridge 16 is of conventional design, comprising a medicine-filled reservoir sealed at one end by a slidable rubber pad 15 and is also sealed at the other end by an injection needle-pierceable septum. Retainer 20 is made of a clear plastic material to hold the contents of the medicine-filled reservoir. Protective cap 17 that removably mounts to the cartridge retainer 20 for protection thereof.

FIG. 5 provides a pen injector which includes a protective external housing 22 is elliptical in transverse cross-section. To facilitate assembly of the device, housing 22 is formed from multiple, interconnected injection molded plastic pieces. Housing 22 is shown having longitudinal halves which included top body or upper housing 23 and bottom body or bottom housing 24 that are complementarily designed to be mate and be fixedly secured together during manufacture by conventionally known methods, such as via ultrasonic welding or adhesive.

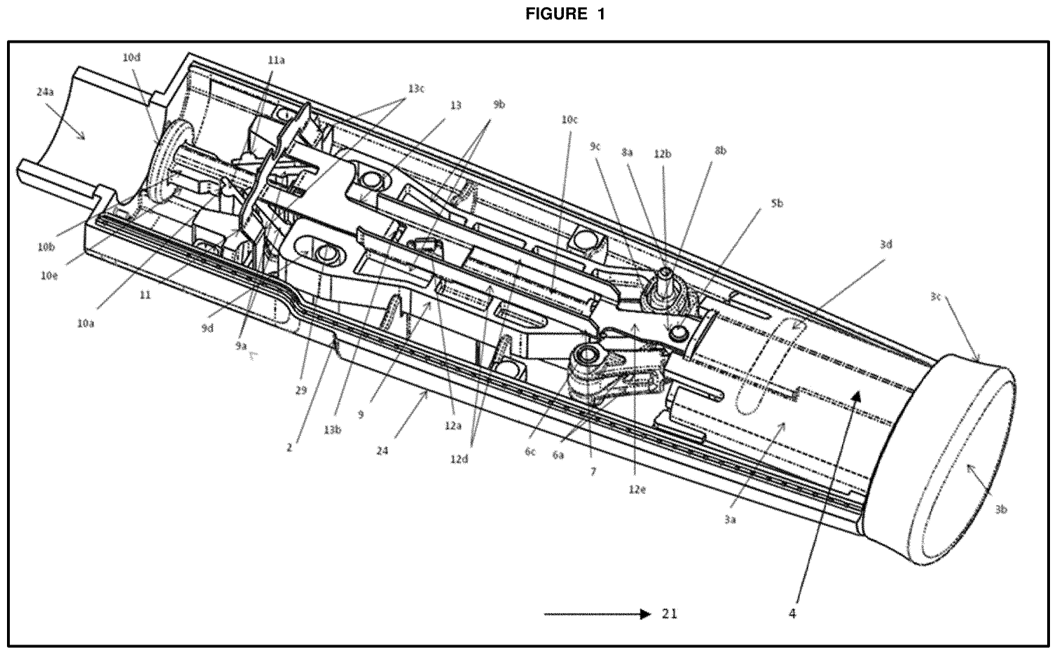

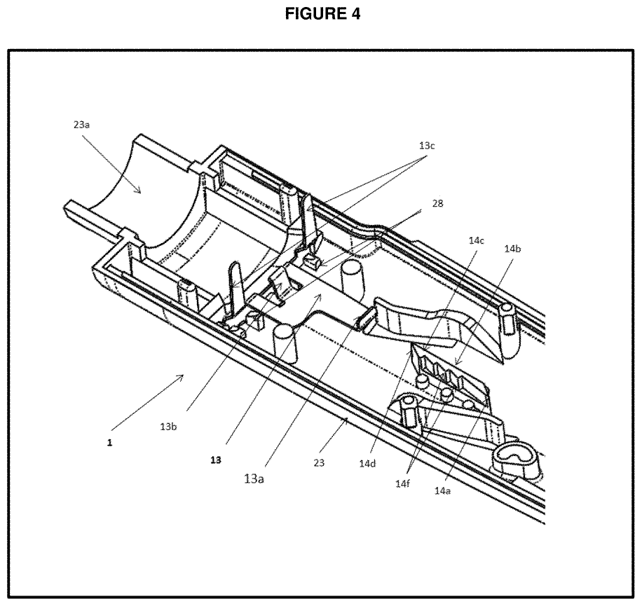

FIG. 4 provides interior surfaces of upper housing 1 and FIG. 1 provides interior surfaces of bottom housing 2. The upper housing halve 23 shown under FIG. 4 and lower housing halve 24 shown under FIG. 5 are formed with a variety of ribs and bulkheads that serve to maintain the alignment and lead the motion of the device components disposed within housing 22. Housing halves 23 and 24 respectively include distally projecting, curved flanges 23a and 24a as shown under FIG. 5. During device manufacture, in order to mount the fluid container to the assembled housing, flanges 23a and 24a are first inserted within the retainer 20 radially outward of the cartridge body, and then fixedly secured to the retainer. When retainer 20 and housing 22 are so secured, cartridge 16 is axially sandwiched between the interior surface of retainer 20 and an internal bulkhead of the housing to prevent axial movement of the cartridge during use.

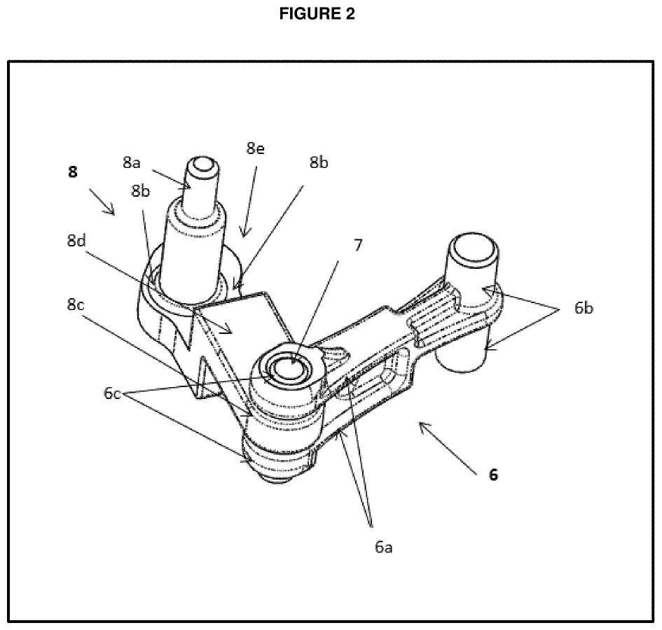

FIG. 1 provides a pictorial representation of a pen injector and the introduction on components of drive member. Pen proximal portion 21 (shown in FIG. 5) includes an axially advanceable drive member 26 (referred as 9, 10, 11 & 27) which includes reducer arm set 27 (referred as 5, 6, 7 & 8 in FIGS. 2 & 3). Drive member includes a plunger 10 (referred as 10a-10e in FIG. 1) and a drive ratchet 9 and drive ratchet legs (or resilient pawl) 9a and holding ratchet 11 and holding ratchet leg or pawl 11a. Plunger 10 has a square rod-shaped body 10b with the teeth 10a that extends in the axial direction to a proximal end 10c, and weight or load controlling, disc-shaped portion 10d formed at distal end of body 10e.

FIG. 1 provides a drive member elements 9,10 and 11 are constrained by the interior surfaces of housing halves 23 and 24 to be axially translatable and fixed within the housing. Plunger 10 is movable in the distal direction and prevented from proximal movement relative to the housing halves, while drive ratchet 9 is slidably connected to plunger element 10a to be moveable relative thereto in a proximal direction and distal direction, while holding ratchet 11 fixed at bottom housing 2 with holding ratchet legs or pawl 11a that are slidably attached to plunger element 10a to prevent the proximal movement of plunger 10, this one-way axial motion is achieved with ratchets in device 19. In particular, body 10b of plunger 10 includes a row of one-way ramping ratchet teeth 10a on two opposite sides of its four sides, which teeth continue uninterrupted along a portion of the axial length of the body. Ratchet teeth or plunger teeth 10a are engaged by a pair of diametrically opposed, ratchet leg or pawls 11a integrally fixed with housing half 24. Pawls 11a slide along over teeth 10a there by advancing plunger in distal direction during use, wherein the teeth 10a is locked so that backward movement of the plunger is prevented.

FIG. 1 provides a proximal view of pawls or legs 11a, a pair of diametrically opposed drive ratchet legs or resilient pawls 9a of drive ratchet 9 also engage the same rows of ratchet or plunger teeth 10a on two teeth behind the ratchet legs 11a of body 10b. Resilient pawls 9a slide over one teeth 10a when moved proximally during pen cocking and pawls 11a slide over one teeth 10a during the distal advancement of drive ratchet 9 during injection, thereby plunger 10 gets locked by the holding ratchet legs or pawl 11a for proximal movement. The pitch or distance between the transverse face of each adjacent tooth 10a preferably is the distance rubber pad 15 needs to be advanced to deliver the fixed dose of drug.

In addition to its resilient pawls 9a, drive ratchet 9 contains two parallel limbs or arms 9b with the bore which engages with two upward extending protrusions 29 of integrally made in bottom housing 2 at the junction of these two arms 9c to engage with reducer arm, further these two arms 9b consists of two separate bores 9d to engage with the bottom housing 2, drive ratchet member 9b receives slidable rod shaped plunger body 10b.

Button 3 (or Dose knob Thumb-pad) is molded from plastic, externally sized and shaped to be rotatably fixed while slideable within housing 22. An internal hollow member 3a of button 3 of FIG. 1 accommodates a spring 4 (biasing member) axially extending there through. The proximal end of button 3 is covered with a softer material shown at 3b, which is formed via a process. A manually pullable grip portion 3c of button 3 is covered with the soft touch material and extends proximally of the housing 22. An indicating band 3d on button 3 is visible to a user when the button has been properly withdrawn to prepare pen 19 for medication delivery.

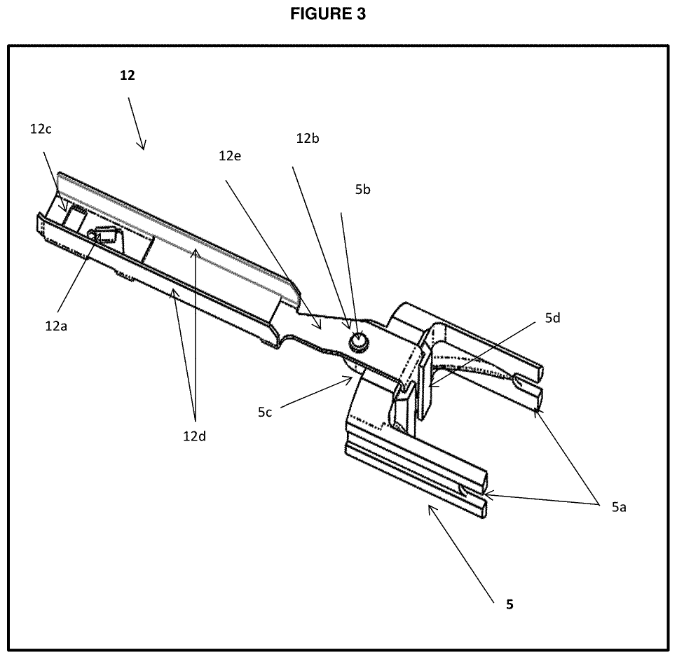

FIG. 3 provides carrier 5, made of injection molded material, which includes two parallel diametrically opposite extending arms 5a is keyed to be fixed within the button 3. Carrier 5 includes a space or slot 5d, this space or slot made to fix or engage with the distal end of the force limiting biasing member 4 made of metal, helically coiled compression spring. The proximal end of biasing member 4 fits or engages to the slot formed in the button 3 within the hollow space 3a. The spring 4 is captured in a pre-stressed between the part of carrier engages with the distal part of spring 5d and the slot to fit the proximal end of spring in the button, which pre-stressing is at least the maximum possible forces that users may apply on the plunger button during dose setting of pen injector. In one of the embodiments, the mechanical advantage of the pre-stressing is in an amount of the spring constant is more than the frictional force, the spring stiffness is larger than the frictional resistance of the parts, so the spring acts as cushion during mechanism jams, so the spring pushes the carrier axially. Coil spring 4 is also designed and made with sufficient spacing in its coiling, and with proper elastic properties, such that the spring, by compression, can accommodate movement of button 3 from the cocked position to the ready-to-be-cocked position without movement of carrier 5, whereby spring 4 can absorb plunging forces that may damage the internal components.

Carrier 5 includes a bore like structure or slot 5c at distal end of carrier 5 to fix or engage with proximal end of fork link 6 at an upward and downward protrusions 6b. At distal end of fork link, it has two parallel opposite extending arms 6a with bores or slots on it 6c to engage it with reducer arm bore or slot 8c by fixing or receiving a pin like structure called rivet 7 in it, which defines an axis about which the reducer arm set pivots during use. A protrusion on the distal end of carrier 5b serve as base to which a partial dose prevention plate or sheet 12 fixed or inserted.

FIG. 3 also describes a partial dose prevention (PDP) plate 12 made up of one piece metal sheet proximally fixed with carrier element 5c. At distal end of sheet or PDP plate 12, a blade like structure 12a integrally made within the PDP plate 12 which slides over or directly engages with a one way track 14 (of FIG. 4) which is integrally made within the upper housing 1 of housing half 23, further PDP plate 12 includes two opposite extending bars proximally from 12c and further it contains metal sheet extending distally from 12b to 12d.

In one embodiment, the present invention provides a dose setting steps of the medication delivery device comprising the following:
(a) pulling the dose knob thumb-pad proximally which in turn pulls the carrier;
(b) the carrier pulls the fork link by sliding in the dose knob;
(c) the fork link pulls the reducer arm by getting riveted with it and pivoted in the carrier and the reducer arm slides over the drive ratchet;
(d) the drive ratchet moves in the proximal direction slipping on the plunger teeth;
(e) the dose knob carrying the part of PDP plate is riveted with the carrier and slides on the one way track of the upper housing;
(f) the plunger which is butting to the rubber pad is held in a position by holding ratchet and bottom housing ribs to be driven forward by the drive ratchet.

In embodiments, the present invention provides a dose setting step of the medication delivery device, wherein the reducer arm set advances proximally and moves a constant distance of “N” mm in proximal direction. The proximal travel distance i.e. “N” mm (between about 10 mm to 30 mm) of Reducer arm set which translates into “n” mm (between about 0.5 to 3 mm) of proximal travel distance of drive ratchet 9 because, the reducer arm member 8b is slidably engaged with the drive ratchet member 9c to slide, which leads to locking of drive ratchet member 9a in plunger member 10a. The reducer arm 8 holds “n” mm distance between the reducer arm member 8a and 8e which gives a movement to drive ratchet 9 proximally to “n” mm distance, and reducer arm member 8e engaged or fixed in slot 30 made within the bottom housing 2 is an integral part of the device.

In certain embodiments, the present invention provides a dose setting step of the medication delivery device, wherein the reducer arm set advances proximally and moves a constant distance of 17 mm in proximal direction. The proximal travel distance (i.e. 17 mm) of Reducer arm set translates into 1.1 mm proximal travel distance of drive ratchet 9 because, the reducer arm member 8b is slidably engaged with the drive ratchet member 9c to slide, which leads to locking of drive ratchet member 9a in plunger member 10a. The reducer arm 8 holds 1.1 mm distance between the reducer arm member 8a and 8e which gives a movement to drive ratchet 9 proximally to 1.1 mm distance, and reducer arm member 8e engaged or fixed in slot 30 made within the bottom housing 2 is an integral part of the device

In embodiments, the present invention provides a dose delivery steps of the medication delivery device comprising the following:
(a) pushing the dose knob thumb-pad towards distal direction to the fully shut position;
(b) the carrier attached to the thumb-pad pushes the fork link which pushes the reducer arm;
(c) the reducer arm slides through the drive ratchet and pushes the drive ratchet to move one teeth forward at the end of dose knob thumb pad stroke;
(d) the drive ratchet gets locked in the plunger teeth and moves the plunger forward;
(e) the plunger moves forward by slipping on the holding ratchet and moves the rubber pad forward which pushes the cartridge to deliver the predetermined dose of the drug.

In embodiments, the present invention provides a dose delivery step of the medication delivery device, wherein the reducer arm set advances distally and pushes the plunger 10 distally, whereas reducer arm set along with button assembly 3 moves a constant distance “M”mm (between about 10 mm to 30 mm) in distal direction. Reducer arm 8 is utilized in the device to convert the “M”mm distal distance of reducer arm set to translate into “m” mm (between about 0.5 to 3 mm) travel of drive ratchet 9 distally because, the reducer arm member 8b is slidably engaged with the drive ratchet member 9c to slide, which leads to move drive ratchet member 9a along with plunger member 10a distally and thereby leads plunger to move forward axially.

In certain embodiments, the present invention provides a dose delivery steps of the medication delivery device the reducer arm set which advances axially and pushes the plunger 10 distally, whereas reducer arm set along with button assembly 3 moves a constant distance (i.e. 17 mm) in distal direction. Reducer arm 8 is utilized in the device to convert the 17 mm proximal distance of reducer arm set to translate into 1.1 mm travel of drive ratchet 9 proximally because, the reducer arm member 8b is slidably engaged with the drive ratchet member 9c to slide which leads to locking of drive ratchet member 9a in plunger member 10a and plunger moves forward.

In one embodiment the partial dose prevention comprises movement of a partial dose prevention blade component 12a which slides over the one way track 14 of the upper housing 1 during the dose setting and allows the dose knob thumb-pad to move proximally only and thereby prevents its movement in distal direction.

After the completion of final dose the medication delivery device (pen injector) includes an EOL component which functions as a locking mechanism (or a mechanism with latch lever locking feature) that prevents/stops the proximal or distal movement of the plunger, thereby prevents the device for dose setting and/or dose delivery after the final dose is delivered to the patient. After final dose the locking mechanism automatically operates to prevent the thumb pad or button 3 to move proximally to indicate that user cannot use the device further and hence the device should be disposed off.

FIG. 4 provides a EOL sheet 13 made up of metal that includes a lever protrusion 13a at the proximal end, a downward snapping element 13b at distal end, two downward protrusion arms 13c formed by bending the metal sheet at distal end and two protrusions 28 formed integrally during manufacturing inside the upper housing 1 to hold the EOL component, further locking mechanism includes EOL or lever engaging member 12c of the PDP plate 12 (shown under FIG. 3).

The locking mechanism automatically operates during the final dose. As plunger moves axially for final dose, a EOL member 13b which slides over the plunger member 10b losses the contact with plunger member 10b and leads to snap the EOL 13 downward, further EOL member 13c fixes or engages with bottom housing 2. As EOL 13 snaps downward, it is latched or held proximally at EOL member 13a by PDP plate member 12c. This mechanism prevents the button 3 to pull proximally for further dose setting; hence, user cannot use the device further and the device should be disposed off.

In certain embodiments, one way track 14 of FIG. 4 is integrally formed with the interior surface 1 of housing half 23 and includes a bar portion 14c having an angled distal end 14a and angled proximal end 14d. One longitudinally extending face of bar portion 14c provides a flat or plane travel surface 14b, and the opposite face of the bar portion 14c includes a travel surface 14e equipped with a plurality of ratchet teeth 14f. Ratchet teeth 14f are engageable by PDP plate element 12a which slides on teeth 14f while preparing a dose and prevent the distal movement of button 3 on partial pulling since reducer arm set and button 3 assembly together needs to travel to 17 mm proximally for dose setting thereby leads to prevention of distal movement of plunger before complete preparation or setting of dose for injection.

In certain embodiments, the partial dose preventive mechanism provides an initial reluctance to pen cocking due to sliding of PDP plate element 12a over a distal end one way track 14a, along with a prevention of distal movement of reducer arm set, button 3 and drive ratchet assembly 9 prior to the complete dose preparation (dose setting), due to the movement of PDP plate element 12a over the row of teeth 14f of one way track.

The term “medicament”, as used herein, means a pharmaceutical formulation containing at least one pharmaceutically active compound, wherein the pharmaceutically active compound can be a hormone, a peptide, a protein, a polysaccharide, a vaccine, an enzyme, an antibody or an oligonucleotide, or a mixture thereof.

In certain embodiments, the medicaments in the injection device of the present invention can be used to inject a wide range of drugs. For example, injection device can be used to inject drugs, water soluble medicaments and oil soluble medicaments. Some medicaments that can be used with injector device include parathyroid hormone (“PTH”) like teriparatide and various other medications such as exenatide and the like. Injection device can also be used to inject medicaments listed in the Physicians' Desk Reference, 67th Edition (2013) (which is herein incorporated by reference in its entirety), and, without limitation, allergens, amebicides and trichomonacides, amino acid preparations, analeptic agents, analgesics, analgesics/antacids, anesthetics, anorexics, antacids, antihelmintics, antialcohol preparations, antiarthritics, antiasthma agents, antibacterials and antiseptics, antibiotics, antiviral antibiotics, anticancer preparations, anticholinergic drug inhibitors, anticoagulants, anticonvulsants, antidepressants, antidiabetic agents, antidiarrheals, antidiuretics, antienuresis agents, antifibrinolytic agents, antifibrotics (systemic), antiflatulents, antifungal agents, antigonadotropin, antihistamines, antihyperammonia agents, anti-inflammatory agents, antimalarials, antimetabolites, antimigraine preparations, antinauseants, antineoplastics, anti-obesity preparations, antiparasitics, anti-parkinsonism drugs, antipruritics, antipyretics, antispasmodics and antichloinergics, antitoxoplasmosis agents, antitussives, antivertigo agents, antiviral agents, apomorphine, atropine, biologicals, biosimilars, bismuth preparations, bone metabolism regulators, bowel evacuants, bronchial dilators, calcium preparations, cardiovascular preparations, central nervous system stimulants, cerumenolytics, chelating agents, choleretics, cholesterol reducers and anti-hyperlipemics, colonic content acidifiers, cough and cold preparations, decongestants, diazepam, dihydroergotamine, epinephrine expectorants and combinations, diuretics, emetics, enzymes and digestants, fertility agents, fluorine preparations, galactokinetic agents, general anesthetic, geriatrics, germicides, glucagon, haloperidol, hematinics, hemorrhoidal preparations, histamine H receptor antagonists, hormones, hydrocholeretics, hyperglycemic agents, hypnotics, immunosuppressives, laxatives, lovenox, mucolytics, muscle relaxants, narcotic antagonists, narcotic detoxification agents, ophthalmological osmotic dehydrating agents, otic preparations, oxytocics, parashypatholytics, parathyroid preparations, pediculicides, peptide drugs, phosphorus preparations, premenstrual therapeutics, psychostimulants, quinidines, radiopharmaceuticals, respiratory stimulants, salt substitutes, scabicides, sclerosing agents, sedatives, sumatriptan, sympatholytics, sympathomimetics, thrombolytics, thyroid preparations, toradol, tranquilizers, tuberculosis preparations, uricosuric agents, urinary acidifiers, urinary alkalinizing agents, urinary tract analgesic, urological irrigants, uterine contractants, vaginal therapeutics and vitamins and each specific compound or composition listed under each of the foregoing categories in the PDR®. Some other medicaments that can be used with injector device 100 include Ergocalciferol (Calciferol), diethylstilbestrol, Diprovan (propofol), estradiol valerate, fluphenazine decanoate, fulvestrant, intralipid, liposyn, nandrolone decanoate, nebido, nutralipid, paclitaxel, progesterone, prograf, testosterone cypionate, zuclopenthixol, haloperidol dodecanoate, Enbrel, Humira, Lantus, Epogen (Procrit), Neulasta, Aranesp, Avonex, PEGasys, Rebif, Neupogen, Betaseron, Avastin, Remicade, Herceptin, Erbitux, Recombinate, Cerezyme, NovoSeven, Tysabri, Synagis, Copaxone and Kogenate FS. In certain embodiments, the medicament is dissolved in soybean oil, ethyl oleate, castor oil, sesame oil, safflower oil, arachis oil, polyoxyyethylated castor oil (Cremophor® EL), polyoxyl 60 hydrogenated castor oil (HCO-60), cottonseed oil, or thin oil derived from coconut oil.

In some embodiments, the medicament may be a hazardous agent. “Hazardous Agent(s)” as used herein means any one or more medications that are toxic agents, cytotoxic agents and/or other dangerous agents that may cause serious effects upon contact with a subject as well as highly potent agents, agents that have profound physiological effects at low doses. Exemplary hazardous agents include, without limitation, analgesics, immunomodulating agents, IL-1 receptor antagonists, IL-2 alpha receptor antagonists, anti-rejection compounds, hormonal agents, prostaglandins, sedatives, anticholinergic agents, Parkinsons disease drugs, expensive agents, neuroleptic agents, tissue necrosis factor (TNF) blockers, and other dangerous agents. Examples of hazardous agents suitable for use with the injection device 100 in accordance with the present invention include, but are not limited to, those disclosed in U.S. Patent Application Publication No. 2012/0157965 entitled “Hazardous Agent Injection System” (to Paul Wotton et. al, published Jun. 21, 2012), which is incorporated by reference herein in its entirety. Particular examples of cytotoxic agents include, without limitation, 6-mercaptopurine, 6-thioinosinic acid, azathioprine, chlorambucil, cyclophosphamide, cytophosphane, cytarabine, fluorouracil, melphalan, methotrexate, uramustine, anti-cytokine biologicals, cell receptor antagonists, cell receptor analogues, and derivatives thereof. Examples of highly potent agents include, without limitation, steroids such as dexamethasone, progesterone, somatostatin, and analogues thereof; biologically active peptides such as teriparatide; and anticholinergics such as scopolamine. Examples of agents that have profound physiological effects at low doses include, without limitation, antihypertensives and/or blood pressure down regulators. Examples of analgesics include, without limitation, fentanyl, fentanyl citrate, morphine, meperidine, and other opioids. Examples of immunomodulating agents include, without limitation, adalimumab (anti-tissue necrosis factor monoclonal antibody or anti-TNF). Examples of IL-1 receptor antagonists include, without limitation, anakinra. Examples of IL-2 alpha receptor antagonists include, without limitation, daclizumab and basiliximab. Examples of anti-rejection compounds include, without limitation, azathioprine, cyclosporine, and tacrolimus. Examples of hormonal agents include, without limitation, testosterone, estrogen, growth hormone, insulin, thyroid hormone, follicle stimulating hormone (FSH), epinephrine/adrenaline, progesterone, parathyroid hormone, gonadotrophin releasing hormone (GHRH), leutinizing hormone releasing hormone (LHRH), other hormones such as those where contact with the hormone by members of the opposite sex can lead to side effects, and derivatives thereof. Examples of prostaglandins include, without limitation, gamma-linolenic acid, docosahexanoic acid, arachidonic acid and eicosapentaenoic acid. Examples of sedatives include, without limitation, barbiturates such as amobarbital, pentobarbital, secobarbital, and phenobarbitol; benzodiazepines such as clonazepam, diazepam, estazolam, flunitrazepam, lorazepam, midazolam, nitrazepam, oxazepam, triazolam, temazepam, chlordiazepoxide, and alprazolam; herbal sedatives such as ashwagandha, duboisia hopwoodii, prosanthera striatiflora, kava (piper methysticum), mandrake, valerian, and marijuana; non-benzodiazepine sedatives (a.k.a. “Z-drugs”) such as eszopiclone, zaleplon, zolpidem, zopiclone; antihistamines such as diphenhydramine, dimenhydrinate, doxylamine, and promethazine; and other sedatives such as chloral hydrate. Examples of anticholinergic agents include, without limitation, dicyclomine, atropine, ipratropium bromide, oxitropium bromide, and tiotropium. Examples of Parkinson's disease drugs include, without limitation, levodopa, dopamine, carbidopa, benserazide, co-ceraldopa, co-beneldopa, tolcapone, entacapone, bromocriptine, pergolide, pramipexole, ropinirole, piribedil, cabergoline, apomorphine, and lisuride. Examples of expensive agents include, without limitation, human growth hormone and erythropoietin. Examples of neuroleptic agents includes, without limitation, antipsychotics; butyrophenones such as haloperidol and droperidol; phenothiazines such as chlorpromazine, fluphenazine, perphenazine, prochlorperazine, thioridazine, trifluoperazine, mesoridazine, periciazine, promazine, triflupromazine, levomepromazine, promethazine, and pimozide; thioxanthenes such as chlorprothixene, clopenthixol, flupenthixol, thiothixene, and zuclopenthixol; atypical antipsychotics such as clozapine, olanzapine, risperidone, quetiapine, ziprasidone, amisulpride, asenapine, paliperidone, iloperidone, zotepine, and sertindole; and third generation antipsychotics such as aripiprazole and bifeprunox. Examples of TNF blockers includes, without limitation, etanercept.

Wherein in a certain embodiment the pharmaceutically active compound comprises at least one hormonal medicament, preferably the hormonal medicament is a parathyroid hormonal medicament.

Wherein in a further embodiment the pharmaceutically active compound is useful for the treatment and/or prophylaxis of osteoporosis, vertebral fractures, non-vertebral fractures, arthritis, rheumatoid arthritis, osteoarthritis, bone loss, hyperthyroidism, diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and the like.

Wherein in a further embodiment the pharmaceutical active compound comprises at least a parathyroid hormone useful for the treatment of osteoporosis, vertebral fractures, non-vertebral fractures, arthritis, rheumatoid arthritis, osteoarthritis, bone loss and the like.

In certain embodiments, a medicament can typically be administered parenterally, preferably by subcutaneous injection, by methods and in formulations well known in the art. Stabilized formulations of the medicament of present invention can include a stabilizing agent, a buffering agent, a preservative, and the like.

The stabilizing agent incorporated into the solution or composition includes a polyol which includes a saccharide, preferably a monosaccharide or disaccharide, e.g., glucose, trehalose, raffinose, or sucrose; a sugar alcohol such as, for example, mannitol, sorbitol or inositol, and a polyhydric alcohol such as glycerine or propylene glycol or mixtures thereof. A preferred polyol is mannitol or propylene glycol. The concentration of polyol may range from about 1 to about 20 wt-%, preferably about 3 to 10 wt-% of the total solution. The buffering agent employed in the solution or composition of the present invention may be any acid or salt combination which is pharmaceutically acceptable and capable of maintaining the aqueous solution at a pH range of 3 to 7, preferably 3-6. Useful buffering systems are, for example, acetate, tartrate or citrate sources. Preferred buffer systems are acetate or tartrate sources, most preferred is an acetate source. The concentration of buffer may be in the range of about 2 mM to about 500 mM, preferably about 2 mM to 100 mM. The stabilized solution or composition of the present invention may also include a parenterally acceptable preservative. Such preservatives include, for example, cresols, benzyl alcohol, phenol, benzalkonium chloride, benzethonium chloride, chlorobutanol, phenylethyl alcohol, methyl paraben, propyl paraben, thimerosal and phenylmercuric nitrate and acetate. A preferred preservative is m-cresol or benzyl alcohol; most preferred is m-cresol. The amount of preservative employed may range from about 0.1 to about 2 wt-%, preferably about 0.3 to about 1.0 wt-% of the total solution.

The medicaments employed for the injection device of the present invention includes PTH preparations which can be reconstituted from fresh or lyophilized hormone, and incorporate various forms of carrier, excipient and vehicle. Most are prepared in water-based vehicles such as saline, or water acidified typically with acetic acid to solubilize the hormone. The majority of reported formulations also incorporate albumin as a stabilizer (see for example Reeve at al., Br. Med. J., 1980, 280:6228; Reeve at al., Lancet, 1976, 1:1035; Reeve at al., Calcif. Tissue Res., 1976, 21:469; Hodsman et al., Bone Miner 1990, 9(2):137; Tsai et al., J. Clin. Endocrinol Metab., 1989, 69(5):1024; Isaac et al., Horm. Metab. Res., 1980, 12(9):487; Law et al., J. Clin Invest. 1983, 72(3):1106; and Hulter, J. Clin Hypertens, 1986, 2(4):360). Other reported formulations have incorporated an excipient such as mannitol, which is present either with the lyophilized hormone or in the reconstitution vehicle. Formulations representative of those employed for human studies include a human PTH(1-34) (SEQ ID NO: 2) preparation consisting, upon reconstitution, of mannitol, heat inactivated human serum albumin, and caproic acid (a protease inhibitor) as absorption enhancer (see Reeve at al., 1976, Calcif. Tissue Res., 21, Suppl., 469-477); a human PTH (1-38) preparation reconstituted into a saline vehicle (see Hodsman et al., 1991, 14(1), 67-83); and a bovine PTH (1-34) preparation in aqueous vehicle pH adjusted with acetic acid and containing albumin. There is also an International Reference preparation which for human PTH (1-84) (SEQ ID NO: 1) consists of 100 ng of hormone ampouled with 250 μg human serum albumin and 1.25 mg lactose (1981), and for bovine PTH (1-84) consists of 10.mu.g lyophilized hormone in 0.01 M acetic acid and 0.1% w/v mannitol (see Martindale, The Extra Pharmacopeia, The Pharmaceutical Press. London, 29th Edition, 1989 at p. 1338).

In certain embodiments, use of the pen injector of the present invention further provides delivery of a liquid medicated formulation comprising but are not limited to 0.25 mg rhPTH, 45.4 mg mannitol, 3 mg m-cresol, 0.41 mg acetic acid and 0.1 mg sodium acetate were mixed into a solution with 1 ml of distilled water and maintained the pH of the solution from about 3 to 6 with sodium hydroxide and hydrochloric acid.

In certain embodiments, the present invention provides pen injectors for a simple, easy, safe and accurate administration of a fixed-dose drug administration of parathyroid hormone like teriparatide administered as a subcutaneous injection into the thigh or abdominal wall as 20 mcg, once a day for up to 28 days.

We claim:

1. A medication delivery device comprising: a housing; a drive member within said housing movable in distal direction and proximal direction; a medicine-filled fluid container with a movable rubber pad at one end and an outlet at the other end, said rubber pad engageable by plunger to be advanced toward said outlet a distance equal to a distal movement of said plunger; wherein the drive member comprising of plunger, holding ratchet, drive ratchet and reducer arm set; a locking element comprising of an End of Life (EOL) member and a partial dose prevention (PDP) plate, wherein the said EOL engaged within the upper housing or within the lower housing, and wherein a part of EOL member sliding over the plunger member, and the said sliding EOL member losses the contact with the sliding surface of the plunger member after the final dose delivery and a component of EOL member snaps downward and latches with the PDP plate and thereby blocks further dosing.

2. The medication delivery device of claim 1 wherein said plunger on top view comprising of double teethed on both sides and a middle flat surface.

3. The medication delivery device of claim 2 wherein one end of said plunger comprising of disc shaped finish which is engageable with a rubber pad.

4. The medication delivery device of claim 1 wherein said reducer arm set comprising of carrier, fork link, reducer arm and rivet.

5. The medication delivery device of claim 1 comprising a partial dose prevention plate.

6. The partial dose prevention plate of claim 6 comprising of rectangular cavity component, blade shaped component and a circular bore.

7. A medication delivery device comprising the step of dose setting, wherein the steps comprises:
(a) pulling the dose knob thumb-pad proximally which in turn pulls the carrier;
(b) the carrier pulls the fork link by sliding in the dose knob;
(c) the fork link pulls the reducer arm by getting riveted with it and pivoted in the carrier and the reducer arm slides over the drive ratchet;
(d) the drive ratchet moves in the proximal direction slipping on the plunger teeth;
(e) the dose knob carrying the part of PDP plate is riveted with the carrier and slides on the one way track of the upper housing;
(f) the plunger which is butting to the rubber pad is held in a position by holding ratchet and bottom housing ribs to be driven forward by the drive ratchet.

8. A medication delivery device comprising the step of dose delivery, wherein the steps comprises:
(a) pushing the dose knob thumb-pad towards distal direction to the fully shut position;
(b) the carrier attached to the thumb-pad pushes the fork link which pushes the reducer arm;
(c) the reducer arm slides through the drive ratchet and pushes the drive ratchet to move one teeth forward at the end of dose knob thumb pad stroke;
(d) the drive ratchet gets locked in the plunger teeth and moves the plunger forward;
(e) the plunger moves forward by slipping on the holding ratchet and moves the rubber pad forward which pushes the cartridge to deliver the predetermined dose of the drug.

9. The partial dose prevention of the medication delivery device of claim 1, comprises movement of a partial dose prevention blade component which slides over the one way track of the upper housing during dose setting and allows the dose knob thumb-pad to move proximally and prevents its movement in distal direction.

10. The medication delivery device of claim 1, comprising liquid pharmaceutical formulation comprising: a) human parathyroid hormone (1-34); b) glacial acetic acid; c) sodium acetate; d) mannitol; e) metacresol; f) purified water; and g) sodium hydroxide and hydrochloric acid to maintain a pH from about 3 to 6.
